(19) United States
(12) Patent Application Publication
(10) Pub. No.: US 2007/0004777 A1
(43) Pub. Date: Jan. 4, 2007

(54) Methods for treating or preventing acute myelogenous leukemia

(76) Inventor: Shripad S. Bhagwat, San Diego, CA (US)

Correspondence Address: JONES DAY, 222 EAST 41ST ST, NEW YORK, NY, 10017, US

(21) Appl. No.: 11/376,786

(22) Filed: Mar. 15, 2006

Related U.S. Application Data
(60) Provisional application No. 60/664,572, filed on Mar. 23, 2005.

(51) Int. Cl. A61K 31/454

(52) U.S. Cl. 514/322

(57) ABSTRACT

This invention is generally directed to methods for treating or preventing acute myelogenous leukemia (“AML”) comprising administering to a patient in need thereof an effective amount of an Indazole Compound having the structure:

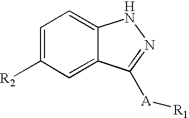

or a pharmaceutically acceptable salt, solvate, hydrate, prodrug, stereoisomer or enantiomer thereof, wherein R₁, R₂ and A are as defined herein.

This application claims the priority benefit of U.S. application No. 60/664,572, filed Mar. 23, 2005, the disclosure of which is incorporated by reference herein in its entirety.

### 1. FIELD OF THE INVENTION

This invention is generally directed to methods for treating acute myelogenous leukemia (“AML”) comprising administering to a patient in need thereof an effective amount of an Indazole Compound or pharmaceutically acceptable salt, solvate, hydrate, prodrug or isomer thereof. Methods for preventing AML comprising administering to a patient in need thereof an effective amount of an Indazole Compound or pharmaceutically acceptable salt, solvate, hydrate, prodrug or isomer thereof are also provided.

### 2. BACKGROUND OF THE INVENTION

The Jun N-terminal kinase (JNK) pathway is activated by exposure of cells to environmental stress or by treatment of cells with pro-inflammatory cytokines. Activation of the JNK pathway has been documented in a number of disease settings, providing the rationale for targeting this pathway for drug discovery. In addition, molecular genetic approaches have validated the pathogenic role of this pathway in several diseases, such as cancer.

Cancer is characterized by uncontrolled growth, proliferation and migration of cells. Cancer is the second leading cause of death with 500,000 deaths and an estimated 1.3 million new cases in the United States in 1996. The role of signal transduction pathways contributing to cell transformation and cancer is a generally accepted concept. The JNK pathway leading to AP-1 appears to play a critical role in cancer. Expression of c-jun is altered in early lung cancer and may mediate growth factor signaling in non-small cell lung cancer (Yin T., Sandhu G., Wolfgang C. D., Burrier A., Webb R. L., Rigel D. F. Hai T., and Whelan J. J. Biol. Chem. 272:19943-19950, 1997). Indeed, over-expression of c-jun in cells results in transformation, and blocking c-jun activity inhibits MCF-7 colony formation (Szabo E., Riffe M., Steinberg S. M., Birrer M. J., Linnoila R. I. Cancer Res. 56:305-315, 1996). DNA-damaging agents, ionizing radiation and tumor necrosis factor activate the JNK pathway. In addition to regulating c-jun production and activity, JNK activation can regulate phosphorylation of p53, and thus can modulate cell cycle progression (Chen T. K., Smith L. M., Gebhardt D. K., Birrer M. J., Brown P. H. Mol. Carcinogenesis 15:215-226, 1996). The oncogene BCR-Abl, associated with t(9,22) Philadelphia chromosome translocation of chronic myelogenous leukemia, activates JNK and leads to transformation of hematopoietic cells (Milne D. M., Campbell L. E., Campbell D. G., Meek D. W. J. Biol. Chem. 270:5511-5518, 1995). Selective inhibition of JNK activation by a naturally occurring JNK inhibitory protein, called JIP-1, blocks cellular transformation caused by BCR-Abl expression (Raitano A. B., Halpern J. R., Hambuch T. M., Sawyers C. L. Proc. Nat. Acad. Sci USA 92:11746-11750, 1995). Thus, JNK inhibitors may block transformation and tumor cell growth.

Myeloproliferative disorders (MPDs) are generally caused by acquired clonal abnormalities of the hematopoietic stem cell and include polycythemia vera, myelofibrosis, essential thrombocytosis and chronic myeloid leukemia. C. A. Linker, Blood, in CURRENT MEDICAL DIAGNOSIS & TREATMENT 2002 535 (41^{st} ed. 2002). Symptoms associated with MPDs include, but are not limited to, headache, dizziness, tinnitus, blurred vision, fatigue, night sweat, low-grade fever, generalized pruritus, epistaxis, blurred vision, splenomegaly, abdominal fullness, thrombosis, increased bleeding, anemia, splenic infarction, severe bone pain, hematopoiesis in the liver, ascites, esophageal varices, liver failure, respiratory distress, and priapism.

Abnormalities associated with MPDs include, but are not limited to, clonal expansion of a multipotent hematopoietic progenitor cell with the overproduction of one or more of the formed elements of the blood (e.g., elevated red blood cell count, elevated white blood cell count, and/or elevated platelet count), presence of Philadelphia chromosome or bcr-abl gene, teardrop poikilocytosis on peripheral blood smear, leukoerythroblastic blood picture, giant abnormal platelets, hypercellular bone marrow with reticular or collagen fibrosis, excessive expression of inflammatory cytokines including, but not limited to, TNF-α, IL-1, IL-2 and IL-6, excessive expression of inflammation related enzymes including, but not limited to, iNOS (inducible nitric oxide synthase) and COX-2, and marked left-shifted myeloid series with a low percentage of promyelocytes and blasts.

Accordingly, there is a need in the art for compounds effective for treating and preventing MPDs, such as acute myelogenous leukemia. The present invention fulfills these needs, and provides further related advantages.

### 3. SUMMARY OF THE INVENTION

The present invention relates to methods for treating or preventing acute myelogenous leukemia (“AML”), comprising administering to a patient in need thereof an effective amount of a compound of the invention, or a pharmaceutically acceptable salt, solvate, hydrate, stereoisomer or enantiomer thereof. The present invention further relates to methods for preventing AML, comprising administering to a patient in need thereof an effective amount of a compound of the invention, or a pharmaceutically acceptable salt, solvate, hydrate, stereoisomer or enantiomer thereof.

The compounds of the invention have the following general formula (I): 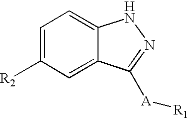
wherein A, R₁ and R₂ are as defined below, including pharmaceutically acceptable salts, solvates, hydrates, prodrugs, stereoisomers and enantiomers thereof. Compounds of formula (I) are set forth in U.S. Pat. No. 6,897,231 B2, issued May 24, 2005, and in International Publication WO 02/10137, published Feb. 7, 2002, both of which are incorporated by reference herein in their entirety.

Compounds of formula (I), or pharmaceutically acceptable salts, solvates, hydrates, prodrugs, stereoisomers and enantiomers thereof, are hereinafter referred to as an “Indazole Compound(s).”

In one embodiment, the invention relates to methods for treating AML comprising administering to a patient in need thereof an effective amount of an Indazole Compound, or a pharmaceutically acceptable salt, solvate, hydrate, prodrug, stereoisomer or enantiomer thereof.

In another embodiment, the invention relates to methods for preventing AML comprising administering to a patient in need thereof an effective amount of an Indazole Compound, or a pharmaceutically acceptable salt, solvate, hydrate, prodrug, stereoisomer or enantiomer thereof.

In another embodiment, the invention relates to methods for treating AML comprising administering to a patient in need thereof a pharmaceutical composition comprising an effective amount of an Indazole Compound, or a pharmaceutically acceptable salt, solvate, hydrate, prodrug, stereoisomer or enantiomer thereof.

In another embodiment, the invention relates to methods for preventing AML comprising administering to a patient in need thereof a pharmaceutical composition comprising an effective amount of an Indazole Compound, or a pharmaceutically acceptable salt, solvate, hydrate, prodrug, stereoisomer or enantiomer thereof.

In another embodiment, the present methods for treating or preventing AML further comprise the administration of an effective amount of another therapeutic agent useful for treating or preventing AML. In this embodiment, the time in which the therapeutic effect of the other therapeutic agent is exerted overlaps with the time in which the therapeutic effect of the Indazole Compound is exerted.

These and other aspects of this invention will be evident upon reference to the following detailed description. To that end, certain patent and other documents are cited herein to more specifically set forth various aspects of this invention. Each of these documents are hereby incorporated by reference in their entirety.

### 4. DETAILED DESCRIPTION OF THE INVENTION

As mentioned above, the present invention is directed to methods for treating or preventing AML comprising administering to a patient in need thereof an effective amount of an Indazole Compound, or a pharmaceutically acceptable salt, solvate, hydrate, prodrug, stereoisomer or enantiomer thereof. The present invention is further directed to methods for preventing AML comprising administering to a patient in need thereof an effective amount of an Indazole Compound, or a pharmaceutically acceptable salt, solvate, hydrate, prodrug, stereoisomer or enantiomer thereof. In certain embodiments, the methods and compositions described herein comprise the use of the free base of an Indazole Compound.

The Indazole Compounds have the following structure (I): 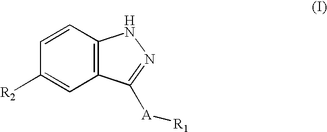
including pharmaceutically acceptable salts, solvates, hydrates, prodrugs, stereoisomers and enantiomers thereof,
wherein:
A is a direct bond, —(CH₂)ₐ—, —(CH₂)_{b}CH═CH(CH₂)_{c}—, or —(CH₂)_{b}C≡C(CH₂)_{c}—;
R₁ is aryl, heteroaryl or heterocycle fused to phenyl, each being optionally substituted with one to four substituents independently selected from R₃;
R₂ is —R₃, —R₄, —(CH₂)_{b}C(═O)OR₅, —(CH₂)_{b}C(═O)OR₅, —(CH₂)_{b}C(═O)NR₅R₆, —(CH₂)_{b}C(═O)NR₅(CH₂)_{c}C(═O)R₆, —(CH₂)_{b}NR₅C(═O)R₆, —(CH₂)_{b}NR₅C(═O)NR₆R₇, —(CH₂)_{b}NR₅R₆, —(CH₂)_{b}OR₅, —(CH₂)_{b}SO_{d}R₅ or —(CH₂)_{b}SO₂NR₅R₆:
a is 1, 2, 3, 4, 5 or 6;
b and c are the same or different and at each occurrence independently selected from 0, 1, 2, 3 or 4;
d is at each occurrence 0, 1 or 2;
R₃ is at each occurrence independently halogen, hydroxy, carboxy, alkyl, alkoxy, haloalkyl, acyloxy, thioalkyl, sulfinylalkyl, sulfonylalkyl, hydroxyalkyl, aryl, substituted aryl, arylalkyl, substituted arylalkyl, heterocycle, substituted heterocycle, heterocycloalkyl, substituted heterocyclealkyl, —C(═O)OR₈, —OC(═O)R₈, —C(═O)NR₈R₉, —C(═O)NR₈OR₉, —SO₂NR₈R₉, —NR₈SO₂R₉, —CN, —NO₂, —NR₈R₉, —NR₈C(═O)R₉, —NR₈C(═O)(CH₂)_{b}OR₉, —NR₈C(═O)(CH₂)_{b}R₉, —O(CH₂)_{b}NR₈R₉, or heterocycle fused to phenyl;
R₄ is alkyl, aryl, arylalkyl, heterocycle or heterocycloalkyl, each being optionally substituted with one to four substituents independently selected from R₃, or R₄ is halogen or hydroxy;
R₅, R₆ and R₇ are the same or different and at each occurrence independently hydrogen, alkyl, aryl, arylalkyl, heterocycle or heterocycloalkyl, wherein each of R₅, R₆ and R₇ are optionally substituted with one to four substituents independently selected from R₃; and
R₈ and R₉ are the same or different and at each occurrence independently hydrogen, alkyl, aryl, arylalkyl, heterocycle, or heterocycloalkyl, or R₈ and R₉ taken together with the atom or atoms to which they are bonded form a heterocycle, wherein each of R₈, R₉, and R₈ and R₉ taken together to form a heterocycle are optionally substituted with one to four substituents independently selected from R₃.

In one embodiment, the Indazole Compounds are those wherein:

when A is a direct bond and R₁ is phenyl,
R₂ is not methyl, methoxy, C(═O)CH₃ or C(═O)H;

when A is a direct bond and R₁ is 4-Me-phenyl,
R₂ is not methyl;

when A is a direct bond and R₁ is 4-F-phenyl,
R₂ is not trifluoromethyl;

when A is a direct bond or —C≡C—and R₁ is phenyl,
R₂ is not —COOEt; and

when A is a direct bond and R₁ is 6,7-dimethoxyisoquinolin-1-yl,
R₂ is not hydroxy.

In one embodiment, -A-R₁ is phenyl, optionally substituted with one to four substituents independently selected from halogen, alkoxy, —NR₈C(═O)R₉, —C(═O)NR₈R₉, and —O(CH₂)_{b}NR₈R₉, wherein b is 2 or 3 and wherein R₈ and R₉ are defined above.

In another embodiment, R₂ is —R₄, —(CH₂)_{b}C(═O)R₅, —(CH₂)_{b}C(═O)OR₅, —(CH₂)_{b}C(═O)NR₅R₆, —(CH₂)_{b}C(═O)NR₅(CH₂)_{c}C(═O)R₆, —(CH₂)_{b}NR₅C(═O)R₆, —(CH₂)_{b}NR₅C(═O)NR₆R₇, —(CH₂)_{b}NR₅R₆, —(CH₂)_{b}OR₅, —(CH₂)_{b}SO_{d}R₅ or —(CH₂)_{b}SO₂NR₅R₆, and b is an integer ranging from 0-4.

In another embodiment, R₂ is —(CH₂)_{b}C(═O)NR₅R₆, —(CH₂)_{b}NR₅C(═O)R₆, 3-triazolyl or 5-tetrazolyl, wherein b is 0 and wherein R₈ and R₉ are defined above.

In a preferred embodiment, R₂ is 3-triazolyl or 5-tetrazolyl.

In another preferred embodiment:

(a) -A-R₁ is phenyl, optionally substituted with one to four substituents independently selected from halogen, alkoxy, —NR₈C(═O)R₉, —C(═O)NR₈R₉, and —O(CH₂)_{b}NR₈R₉, wherein b is 2 or 3; and

(b) R₂ is —(CH₂)_{b}C(═O)NR₅R₆, —(CH₂)_{b}NR₅C(═O)R₆, 3-triazolyl or 5-tetrazolyl, wherein b is 0 and wherein R₅, R₆, R₈ and R₉ are defined above.

In a more preferred embodiment:

(a) -A-R₁ is phenyl, optionally substituted with one to four substituents independently selected from halogen, alkoxy, —NR₈C(═O)R₉, —C(═O)NR₈R₉, and —O(CH₂)_{b}NR₈R₉, wherein b is 2 or 3; and

(b) R₂ is 3-triazolyl or 5-tetrazolyl.

In another preferred embodiment, R₂ is R₄, and R₄ is 3-triazolyl, optionally substituted at its 5-position with:

(a) a C₁-C₄ straight or branched chain alkyl group optionally substituted with a hydroxyl, methylamino, dimethylamino or 1-pyrrolidinyl group; or

(b) a 2-pyrrolidinyl group.

In a more preferred embodiment, R₂ is R₄, and R₄ is 3-triazolyl, optionally substituted at its 5-position with methyl, n-propyl, isopropyl, 1-hydroxyethyl, 3-hydroxypropyl, methylaminomethyl, dimethylaminomethyl, 1-(dimethylamino)ethyl, 1-pyrrolidinylmethyl or 2-pyrrolidinyl.

In one embodiment, an Indazole Compound has structure (II) when A is a direct bond, and has structure (III) when A is —(CH₂)ₐ—:

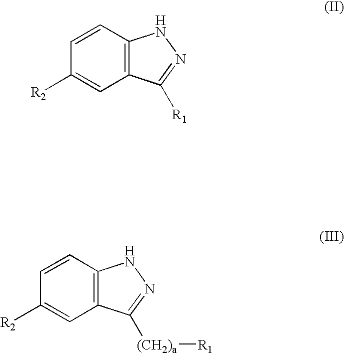

In other embodiments, an Indazole Compound has structure (IV) when A is a —(CH₂)_{b}CH═CH(CH₂)_{c}—, and has structure (V) when A is —(CH₂)_{b}C≡C(CH₂)_{c}—:

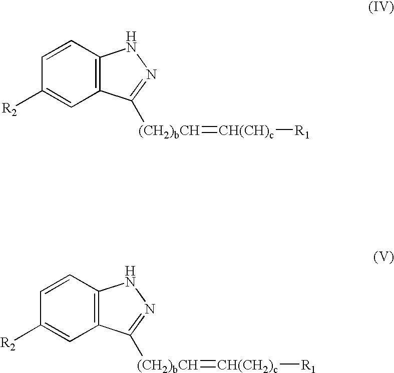

In further embodiments of this invention, R₁ is aryl or substituted aryl, such as phenyl or substituted phenyl as represented by the following structure (VI):

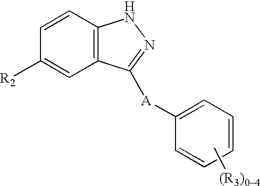

In another embodiment, R₂ is —(CH₂)_{b}NR₄(C═O)R₅. In one aspect of this embodiment, b=0 and an Indazole Compound has the following structure (VII):

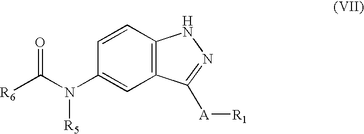

Representative R₂ groups include alkyl (such as methyl and ethyl), halo (such as chloro and fluoro), haloalkyl (such as trifluoromethyl), hydroxy, alkoxy (such as methoxy and ethoxy), amino, arylalkyloxy (such as benzyloxy), mono- or di-alkylamine (such as —NHCH₃, —N(CH₃)₂ and —NHCH₂CH₃), —NHC(═O)R₄ wherein R₆ is a substituted or unsubstituted phenyl or heteroaryl (such as phenyl or heteroaryl substituted with hydroxy, carboxy, amino, alkylester, alkoxy, alkyl, aryl, haloalkyl, halo, —CONH₂ and —CONH alkyl), —NH(heteroarylalkyl) (such as —NHCH₂(3-pyridyl), —NHCH₂(4-pyridyl), heteroaryl (such as pyrazolo, triazolo and tetrazolo), —C(═O)NHR₆ wherein R₆ is hydrogen, alkyl, or as defined above (such as —C(═O)NH₂, —C(═O)NHCH₃, —C(═O)NH(H-carboxyphenyl), —C(═O)N(CH₃)₂), arylalkenyl (such as phenylvinyl, 3-nitrophenylvinyl, 4-carboxyphenylvinyl), heteroarylalkenyl (such as 2-pyridylvinyl, 4-pyridylvinyl).

Representative R₃ groups include halogen (such as chloro and fluoro), alkyl (such as methyl, ethyl and isopropyl), haloalkyl (such as trifluoromethyl), hydroxy, alkoxy (such as methoxy, ethoxy, n-propyloxy and isobutyloxy), amino, mono- or di-alkylamino (such as dimethylamine), aryl (such as phenyl), carboxy, nitro, cyano, sulfinylalkyl (such as methylsulfinyl), sulfonylalkyl (such as methylsulfonyl), sulfonamidoalkyl (such as —NHSO₂CH₃), —NR₈C(═O)(CH₂)_{b}OR₉ (such as —NHC(═O)CH₂OCH₃), NHC(═O)R₉ (such as —NHC(═O)CH₃, —NH(═O)CH₂C₆H₅, —NHC(═O)(2-furanyl)), and —O(CH₂)_{b}NR₈R₉ (such as —O(CH₂)₂N(CH₃)₂).

In another embodiment, the Indazole Compound has the structure (VIII): 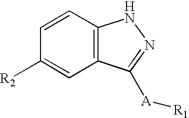
including isomers, prodrugs and pharmaceutically acceptable salts thereof,

wherein:
A is a direct bond, —(CH₂)ₐ—, —(CH₂)_{b}CH═CH(CH₂)_{c}—, or —(CH₂)_{b}C≡C(CH₂)_{c}—;
R₁ is aryl, heteroaryl or heterocycle fused to phenyl, each being optionally substituted with one to four substituents independently selected from R₃;
R₂ is —R₃, —R₄, —(CH₂)_{b}C(═O)R₅, —(CH₂)_{b}C(═O)OR₅, —(CH₂)_{b}C(═O)NR₅R₆, —(CH₂)_{b}C(═O)NR₅(CH₂)_{c}C(═O)R₆, —(CH₂)_{b}NR₅C(═O)R₆, —(CH₂)_{b}NR₅C(═O)NR₆R₇, —(CH₂)_{b}NR₅R₆, —(CH₂)_{b}OR₅, —(CH₂)_{b}SO_{d}R₅ or —(CH₂)_{b}SO₂NR₅R₆;
   a is 1, 2, 3, 4, 5 or 6;
   b and c are the same or different and at each occurrence independently selected from 0, 1, 2, 3 or 4;
   d is at each occurrence 0, 1 or 2;
R₃ is at each occurrence independently —NR₈C(═O)(CH₂)_{b}NR₈R₉;
R₄ is alkyl, aryl, arylalkyl, heterocycle or heterocycloalkyl, each being optionally substituted with one to four substituents independently selected from R₃, or R₄ is halogen or hydroxy;
R₅, R₆ and R₇ are the same or different and at each occurrence independently hydrogen, alkyl, aryl, arylalkyl, heterocycle or heterocycloalkyl, wherein each of R₅, R₆ and R₇ are optionally substituted with one to four substituents independently selected from R₃; and
R₈ and R₉ are the same or different and at each occurrence independently hydrogen, alkyl, aryl, arylalkyl, heterocycle, or heterocycloalkyl, or R₈ and R₉ taken together with the atom or atoms to which they are bonded form a heterocycle, wherein each of R₈, R₉, and R₈ and R₉ taken together to form a heterocycle are optionally substituted with one to four substituents independently selected from R₃.

In a particular embodiment, the Indazole Compound is 1-(5-(1H-1,2,4-triazol-5-yl)(1H-indazol-3-yl))-3-(2-piperidylethoxy)benzene, which has the following structure: 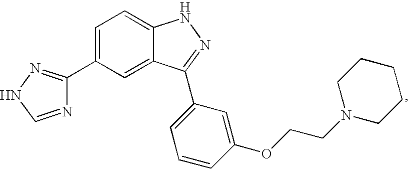
including pharmaceutically acceptable salts, solvates, hydrates, prodrugs and isomers thereof.

In one embodiment, the invention relates to methods for treating AML comprising administering to a patient in need thereof an effective amount of an Indazole Compound, or a pharmaceutically acceptable salt, solvate, hydrate, prodrug, stereoisomer or enantiomer thereof.

In another embodiment, the invention relates to methods for preventing AML comprising administering to a patient in need thereof an effective amount of an Indazole Compound, or a pharmaceutically acceptable salt, solvate, hydrate, prodrug, stereoisomer or enantiomer thereof.

In a particular embodiment, the invention relates to methods for treating AML comprising administering to a patient in need thereof an effective amount of an compound having the structure:

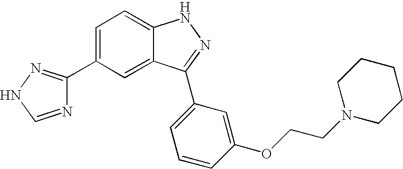

or a pharmaceutically acceptable salt, solvate, hydrate, prodrug, stereoisomer or enantiomer thereof.

In another embodiment, the invention relates to methods for treating AML comprising administering to a patient in need thereof a pharmaceutical composition comprising an effective amount of an Indazole Compound, or a pharmaceutically acceptable salt, solvate, hydrate, prodrug, stereoisomer or enantiomer thereof.

In a particular embodiment, the invention relates to methods for preventing AML comprising administering to a patient in need thereof an effective amount of an compound having the structure:

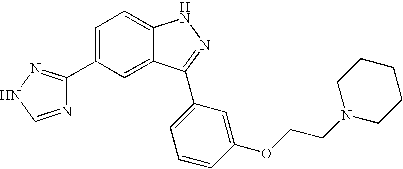

or a pharmaceutically acceptable salt, solvate, hydrate, prodrug, stereoisomer or enantiomer thereof.

In another embodiment, the invention relates to methods for preventing AML comprising administering to a patient in need thereof a pharmaceutical composition comprising an effective amount of an Indazole Compound, or a pharmaceutically acceptable salt, solvate, hydrate, prodrug, stereoisomer or enantiomer thereof.

In another embodiment, the invention relates to methods for treating AML comprising administering to a patient in need thereof a pharmaceutical composition comprising an effective amount of a compound having the structure:

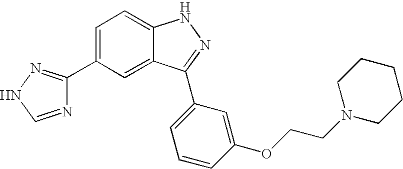

or a pharmaceutically acceptable salt, solvate, hydrate, prodrug, stereoisomer or enantiomer thereof.

In another embodiment, the invention relates to methods for preventing AML comprising administering to a patient in need thereof a pharmaceutical composition comprising an effective amount of a compound having the structure:

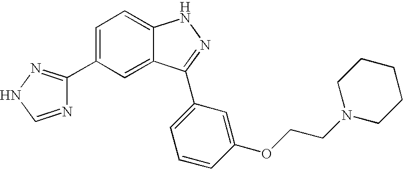

or a pharmaceutically acceptable salt, solvate, hydrate, prodrug, stereoisomer or enantiomer thereof.

In another embodiment, the present methods for treating or preventing AML further comprise the administration of an effective amount of another therapeutic agent useful for treating or preventing AML. In this embodiment, the time in which the therapeutic effect of the other therapeutic agent is exerted overlaps with the time in which the therapeutic effect of the Indazole Compound is exerted.

The terms used herein have following meaning.

“Alkyl” means a straight chain or branched, saturated or unsaturated alkyl, cyclic or non-cyclic hydrocarbon having from 1 to 10 carbon atoms. Representative saturated straight chain alkyls include methyl, ethyl, n-propyl, n-butyl, n-pentyl, n-hexyl, and the like; while saturated branched alkyls include isopropyl, sec-butyl, isobutyl, tert-butyl, isopentyl, and the like. Unsaturated alkyls contain at least one double or triple bond between adjacent carbon atoms (also referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-i butynyl, and the like. Representative saturated cyclic alkyls include cyclopropyl, cyclobutyl, cyclopentyl, cyclohexyl, and the like; while unsaturated cyclic alkyls include cyclopentenyl and cyclohexenyl, and the like. Cycloalkyls are also referred to herein as “carbocyclic” rings systems, and include bi- and tri-cyclic ring systems having from 8 to 14 carbon atoms such as a cycloalkyl (such as cyclopentane or cyclohexane) fused to one or more aromatic (such as phenyl) or non-aromatic (such as cyclohexane) carbocyclic rings.

“Halogen” means fluorine, chlorine, bromine or iodine.

“Keto” means a carbonyl group (i.e., C═O).

“Aryl” means an aromatic carbocyclic moiety such as phenyl or naphthyl.

“Acyloxy means an —OC(O)alkyl group, wherein “alkyl” is defined above.

“Arylalkyl” means an alkyl having at least one alkyl hydrogen atom replaced with an aryl moiety, such as benzyl, —(CH₂)₂phenyl, —(CH₂)₃phenyl, —CH(phenyl)₂, and the like.

“Heteroaryl” means an aromatic heterocycle ring of 5- to 10 members and having at least one heteroatom selected from nitrogen, oxygen and sulfur, and containing at least 1 carbon atom, including both mono- and bicyclic ring systems. Representative heteroaryls are triazolyl, tetrazolyl, oxadiazolyl, pyridyl, furyl, benzofuranyl, thiophenyl, benzothiophenyl, quinolinyl, pyrrolyl, indolyl, oxazolyl, benzoxazolyl, imidazolyl, benzimidazolyl, thiazolyl, benzothiazolyl, isoxazolyl, pyrazolyl, isothiazolyl, pyridazinyl, pyrimidinyl, pyrazinyl, triazinyl, cinnolinyl, phthalazinyl, and quinazolinyl.

“Heteroarylalkyl” means an alkyl having at least one alkyl hydrogen atom replaced with a heteroaryl moiety, such as —CH₂pyridinyl, —CH₂pyrimidinyl, and the like.

“Heterocycle” means a heterocyclic ring containing from 5 to 10 ring atoms

“Heterocycle” means a 5- to 7-membered monocyclic, or 7- to 10-membered bicyclic, heterocyclic ring which is either saturated, unsaturated, or aromatic, and which contains from 1 to 4 heteroatoms independently selected from nitrogen, oxygen and sulfur, and wherein the nitrogen and sulfur heteroatoms can be optionally oxidized, and the nitrogen heteroatom can be optionally quatemized, including bicyclic rings in which any of the above heterocycles are fused to a benzene ring. The heterocycle can be attached via any heteroatom or carbon atom. Heterocycles include heteroaryls as defined above. Thus, in addition to the heteroaryls listed above, heterocycles also include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Heterocycloalkyl” means an alkyl having at least one alkyl hydrogen atom replaced with a heterocycle, such as —CH₂morpholinyl, and the like.

The term “substituted” as used herein means any of the above groups (i.e., aryl, arylalkyl, heterocycle and heterocycloalkyl) wherein at least one hydrogen atom is replaced with a substituent. In the case of a keto substituent, two hydrogen atoms are replaced. Substituents include halogen, hydroxyl, alkyl, substituted alkyl (such as haloalkyl, mono- or di-substituted aminoalkyl, alkyloxyalkyl, and the like), aryl, substituted aryl, arylalkyl, substituted arylalkyl, heterocycle, substituted heterocycle, heterocycloalkyl, substituted heterocycloalkyl, —NRₐR_{b}, —NRₐC(═O)R_{b}, —NRₐC(═O)NRₐR_{b}, —NRₐC(═O)OR_{b}—NRₐSO₂R_{b}, —ORₐ, —C(═O)Rₐ C(═O)ORₐ —C(═O)NRₐR_{b}, —OC(═O)Rₐ, —OC(═O)ORₐ, —OC(═O)NRₐR_{b}, —NRₐSO₂R_{b}, or a radical of the formula -Y-Z-Rₐ where Y is alkanediyl, substituted alkanediyl, or a direct bond, Z is —O—, —S—, —N(R_{b})—, —C(═O)—, —C(═O)O—, —OC(═O)—, —N(R_{b})C(═O)—, —C(═O)N(R_{b})—or a direct bond, wherein Rₐ and R_{b} are the same or different and independently hydrogen, amino, alkyl, substituted alkyl (including halogenated alkyl), aryl, substituted aryl, arylalkyl, substituted arylalkyl, heterocycle, substituted heterocycle, heterocylealkyl or substituted heterocycloalkyl, or wherein Rₐ and R_{b} taken together with the nitrogen atom to which they are attached form a heterocycle or substituted heterocycle.

“Haloalkyl” means alkyl having one or more hydrogen atoms replaced with halogen, such as —CF₃.

“Hydroxyalkyl” means alkyl having one or more hydrogen atoms replaced with hydroxy, such as —CH₂OH

“Sulfonylalkyl” means —SO₂-(alkyl), wherein “alkyl” is defined above;

“Sulfinylalkyl” means —SO—(alkyl), wherein “alkyl” is defined above;

“Thioalky” means —S—(alkyl), wherein “alkyl” is defined above;

“Carboxyl” means —COOH.

“Alkoxy” means —O—(alkyl), wherein “alkyl” is defined above.

An “effective amount” when used in connection with an Indazole Derivative is an amount effective for treating or preventing acute myelogenous leukemia.

The term “hydrate” as used herein means an Indazole Compound that further includes a stoichiometric or non-stoichiometric amount of water bound by non-covalent intermolecular forces.

The term “solvate” as used herein means an Indazole Compound that further includes a stoichiometric or non-stoichiometric amount of a solvent bound by non-covalent intermolecular forces.

The term “prodrug” when used herein means any derivative of an Indazole Compound that is metabolized or otherwise converted into an active form upon introduction into the body of an animal. Prodrugs are well-known to those skilled in the art of pharmaceutical chemistry, and provide benefits such as increased adsorption and half-life. Prodrugs of this invention can be formed when, for example, hydroxy groups are esterified or alkylated, or when carboxyl groups are esterified. Those skilled in the art of drug delivery will readily appreciate that the pharmacokinetic properties of an Indazole Compound can be controlled by an appropriate choice of moieties to produce prodrug derivatives.

Stereoisomers of the Indazole Compounds can be asymmetrically synthesized or resolved using standard techniques such as chiral columns or chiral resolving agents. See, e.g., Jacques, J., et al., Enantiomers, Racemates and Resolutions (Wiley-Interscience, New York, 1981); Wilen, S. H., et al., Tetrahedron 33:2725 (1977); Eliel, E. L., Stereochemistry of Carbon Compounds (McGraw-Hill, NY, 1962); and Wilen, S. H., Tables of Resolving Agents and Optical Resolutions p. 268 (E. L. Eliel, Ed., Univ. of Notre Dame Press, Notre Dame, Ind., 1972).

A “patient” includes an animal (e.g., cow, horse, sheep, pig, chicken, turkey, quail, cat, dog, mouse, rat, rabbit or guinea pig), in one embodiment a mammal such as a non-primate and a primate (e.g., monkey and human), and in another embodiment a human. In certain embodiments, the patient is an infant, child, adolescent or adult. In a particular embodiment, the patient has or is susceptible to having (e.g., through environmental or genetic factors) AML.

The Indazole Compounds can generally be made by organic synthesis techniques known to those skilled in the art, as well as by the following general techniques and by the procedures set forth in the Examples. To that end, the Indazole Compounds can be made according to the following Reaction Schemes 1 through 7 (it should be noted that, in the following reaction schemes, hydrogen atoms are sometimes not depicted and one skilled in organic chemistry would appreciate such accepted shorthand notation):

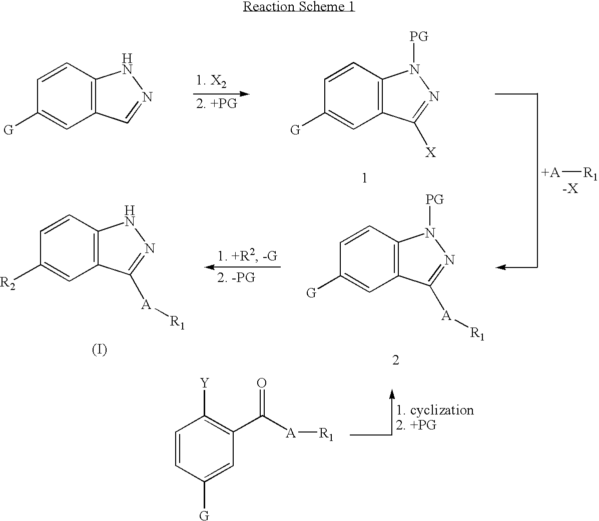

In Reaction Scheme 1, Indazole Compounds can be prepared by techniques well known to those skilled in the art of organic synthesis. Starting from an appropriately 5-substituted indazole, the 3-position can be activated for substitution by use of a suitable dihalogen (X₂). If necessary, a protecting group is then added to the nitrogen at the 1-position (N-1) to give 1. The halogen can be displaced by an appropriately activated A-R₁ moiety to give 2; see, e.g., Reaction Schemes 2 and 5. Alternatively, an appropriately substituted phenyl ketone can be cyclized to give indazole 2 see, e.g., Reaction Schemes 3 and 4. The G moiety can then be left unchanged, displaced or transformed into the desired R₂; see, e.g., Reaction Schemes 3 through 6. Deprotection of N-1 gives indazoles of structure (I).

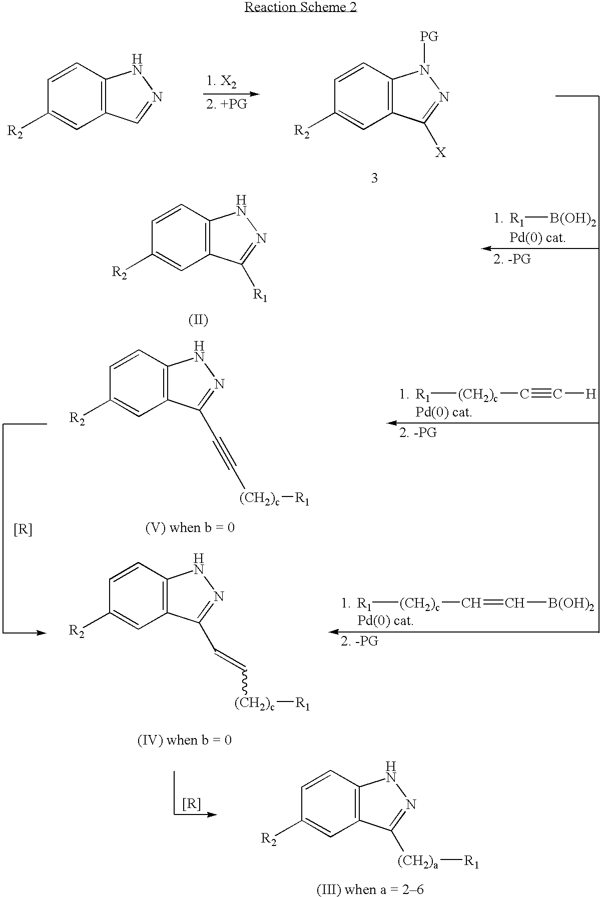

Reaction Scheme 2 illustrates synthetic sequences that yield Indazole Compounds containing various A moieties. Suitable starting materials are commercially available indazoles with the desired R₂ or can be readily prepared, e.g., as in Reaction Schemes 5 and 6. The starting indazole is halogenated at the 3-position with a suitable reagent, e.g., Br₂. It is then protected at N-1 with any suitable nitrogen protecting group to give 3. Suitable protecting groups include but are not limited to acetyl, methoxyethoxymethyl and tetrahydropyranyl. Indazoles, wherein A is a direct bond, can be produced from 3 by displacement of the halogen with an appropriately activated R₁ moiety. For example, in the presence of a suitable Pd(0) or Pd(II) catalyst, R₁-boronic acids can be coupled via a Suzuki reaction to give, after deprotection, compound (II). Analogously, compounds (IV) and (V) can be prepared from suitable alkene and alkyne precursors in the presence of an appropriate Pd(0) catalyst. The cis isomer of indazole (IV) can also be prepared by partial reduction of (V) by, e.g., hydrogenation over BaSO₄ that has been treated with quinoline. Compound (III) can be prepared from (IV) via reduction, e.g., with hydrogen in the presence of Pd—C.

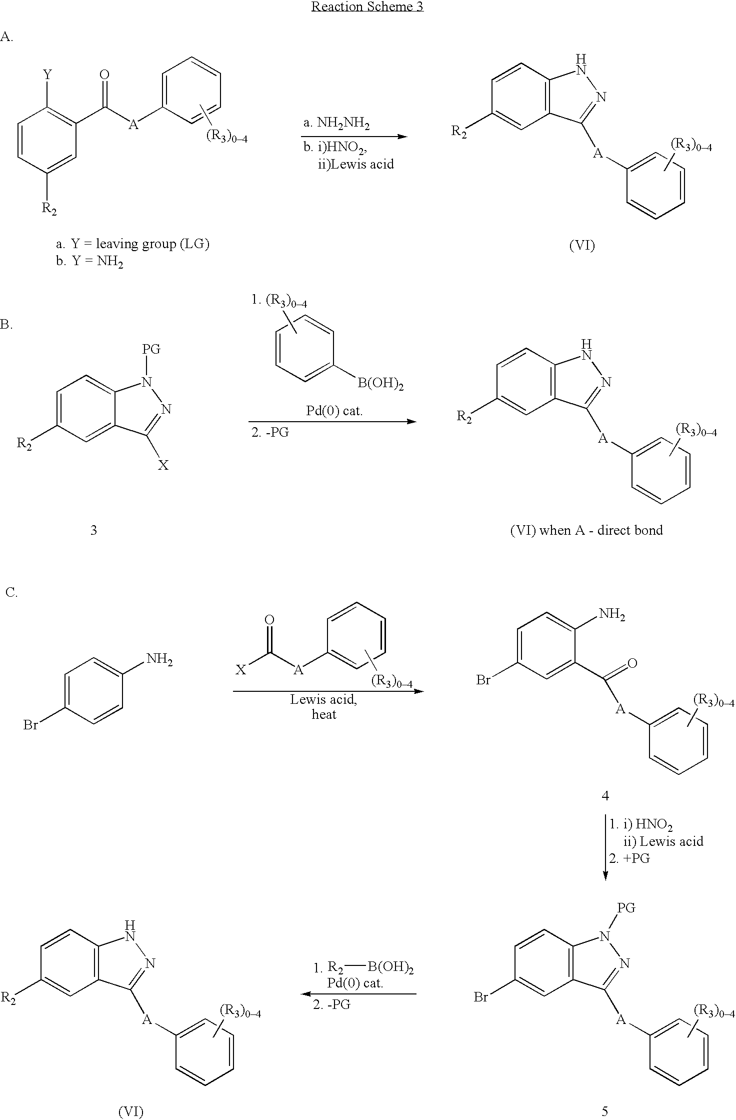

Reaction Scheme 3 illustrates several syntheses of compound (VI) wherein R₁ is depicted as a substituted phenyl group for purposes of illustration only. In Scheme 3A, a phenyl ketone, appropriately substituted at Y and R₂, serves as the starting material. When Y is an amino group, the starting material can be cyclized by exposure, first to HNO₂ and then to a reducing agent, such as SnCl₂, to give compound (VI). Alternatively, when Y is a leaving group such as halogen (e.g., F or Cl), heating the phenyl ketone in the presence of hydrazine effects cyclization to indazole (VI).

In Scheme 3B, halogenated indazole 3 can be coupled with a suitable substituted phenyl moiety and deprotected to give compound (VI), wherein A is a direct bond. By way of example, a phenyl boronic acid substituted with 0-4 R₃ groups will react with a protected 3-bromo-1H-indazole in the presence of a Pd(II) catalyst to yield compound (VI).

Scheme 3C illustrates an alternative synthesis of compound (VI) from the 5-halo-phenyl ketone; this route allows introduction of R₂ groups later in the sequence. 4-Bromo-aniline is acylated with a suitably activated A-R₁ moiety, heated in the presence of an appropriate Lewis acid such as ZnCl₂. For example, a suitably activated A-R₁ group is an acid halide such as carbonyl chloride. The resulting ketone 4 is cyclized as in Scheme 3A, and protected with appropriate groups at the N-1 position as in Scheme 2. The R₂ group can be introduced via a Pd-catalyzed coupling as in Scheme 2, and the protecting group removed to yield compound (VI).

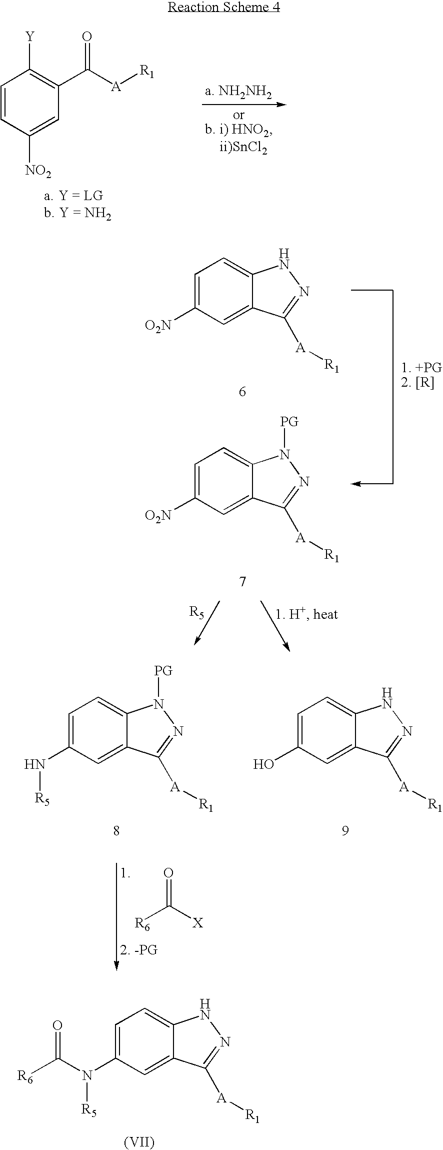

The synthesis of compounds wherein R₂ is an amino carbonyl-containing group is shown by Reaction Scheme 4. In analogy to Scheme 3A, a suitably substituted 4-nitro-phenyl ketone can be cyclized, depending on Y, by exposure either to hydrazine or to HNO₂ and a reducing agent. After protection of N-1, the nitro-group can be reduced by, e.g., hydrogenation over Pd—C, to give 7. The resulting amine can optionally be substituted with R₄, by, e.g., reductive amination, using procedures well known to one skilled in the art of organic synthesis. Compound 8 is acylated with a suitable activated carbonyl moiety and deprotected to give compound (VII). Alternatively, 7 can be hydrolyzed to the 5-hydroxy compound, 9.

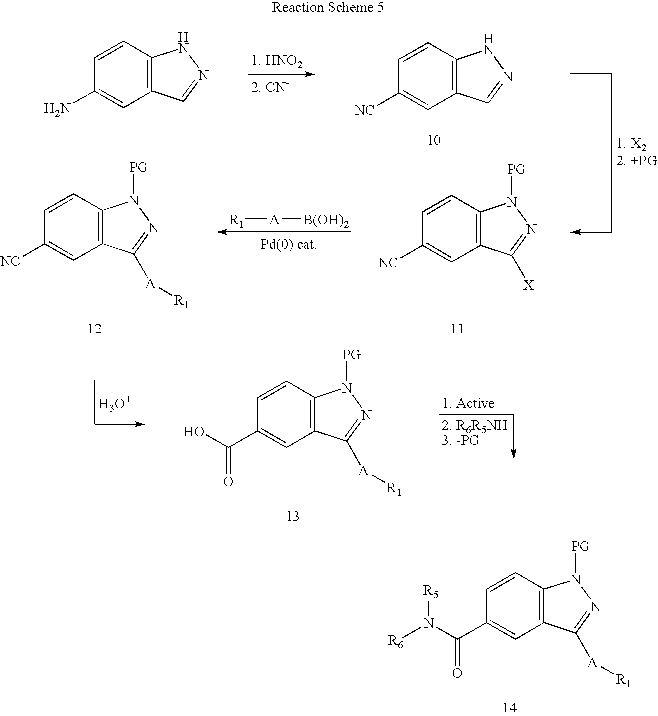

Reaction Scheme 5 illustrates a synthetic route for the preparation of compounds of formula (I) wherein R₂ is a carboxamide. Commercially available 5-amino-1H-indazole is substituted with cyanide at the 5-position to give 10 by treatment with HNO₂, followed, after neutralization to ca. pH 7, by treatment with a cyanide source, e.g., a mixture of CuCn and NaCN. Nitrile 10 can be activated at the 3-position, protected at N-1 and subsequently substituted with an appropriate A-R₁ moiety according to procedures of Scheme 2. The resulting compound, 12, can be hydrolyzed in aqueous acid to give carboxylate 13. Activation of 13 by a suitable method, followed by treatment with R₅R₄NH and deprotection gives the carboxamide, 14. Suitable activation methods include but are not limited to 1) conversion of the carboxylate to an acyl halide (e.g., chloride) and coupling in the presence of pyridine or a related base; and 2) use of a coupling agent suitable for amide bond formation (e.g., dicyclohexylcarbodiimide).

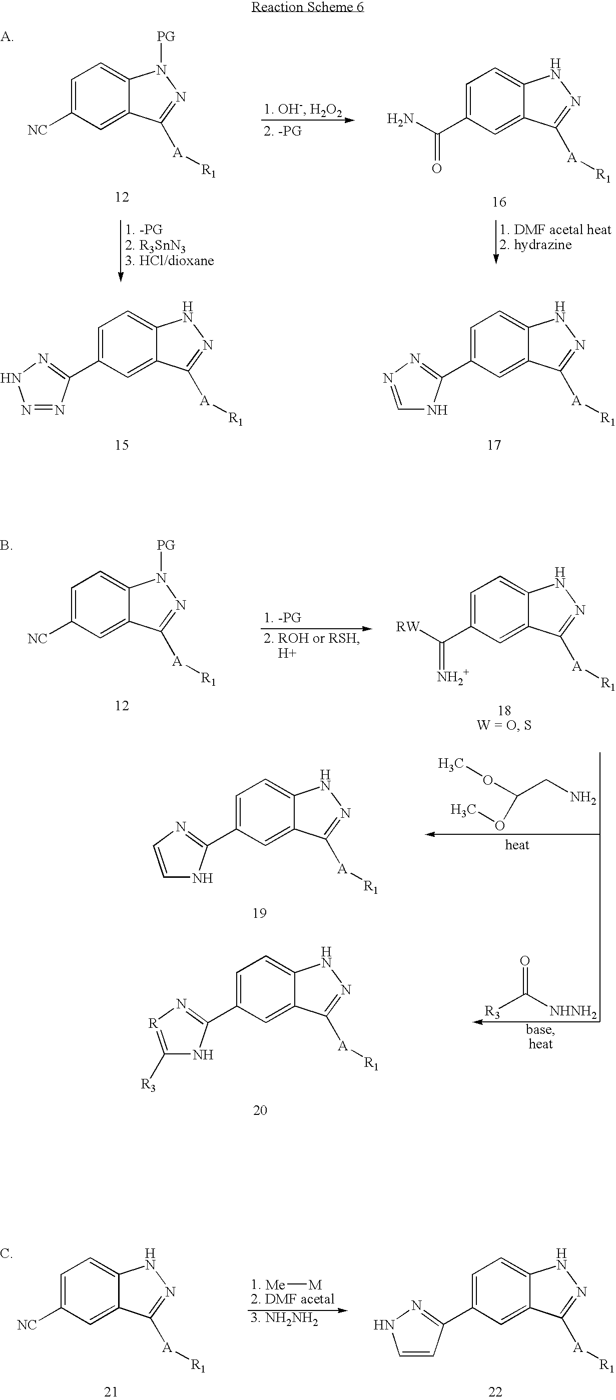

Reaction Scheme 6 illustrates the additional embodiment wherein R₂ is a five-membered heterocyclic substituent. In Scheme 6A, nitrile 12 is deprotected at N-1 and converted to the tetrazole 15 by use of an electrophilic azide source (e.g., a trialkyl tin such as (Bu)₃SnN₃). Nitrile 12 can also be converted to the unsubstituted triazole 17 in four steps. The nitrile is first transformed to the carboxamide by exposure to aqueous base under oxidizing conditions (e.g., NaOH and H₂O₂). The N-1 protecting group is removed to give intermediate 16. The carboxamide is heated with DMF acetal and subsequently treated with hydrazine under acidic conditions to give the desired triazole.

Scheme 6B illustrates the synthesis of imidazole and substituted triazole derivatives at R₂. Nitrile 12 is deprotected and converted to the imidate or thioimidate by heating in the appropriate alcohol or thiol under acidic conditions to give 18. Subsequent exposure to 1-amino-2,2-dimethoxyethane and gentle heating effects formation of imidazole 19. Alternatively, heating 18 with alkyl, aryl or heterocyclic hydrazides under basic conditions (e.g., in presence of a tertiary organoamine such as triethylamine) results in production of 3-substituted triazole 20.

Indazole Compounds can be synthesized according to Scheme 6C. Nitrile 12 can be deprotected at N-1 to give starting material 21. Treatment of the latter nitrile with a suitable organometallic agent, e.g., methyl lithium, yields a methyl ketone intermediate. Subsequent treatment by heating with DMF acetal followed by exposure to hydrazine gives pyrazole 22.

Scheme 7 depicts alternative routes to 5-triazole derivatives of 1H-indazoles. In scheme 7A nitrile 11 is converted to triazole 23 under conditions similar to those employed in Scheme 6B. A suitable protecting group, e.g., trityl, is incorporated onto the free triazole nitrogen to give 24. A-R₁ is then added to position-3 by a boronic acid or other suitable derivative. Finally, the triazole protecting group is removed under, e.g., acidic conditions, to give indazole 17.

In Scheme 7B, starting material 25 is prepared by activation of 13 as, e.g., an acid halide such as chloride. Subsequent reaction with a protected hydrazide followed by removal of protecting groups yields hydrazide 26. By way of example, when PG=acetyl and PG₂=t-butyl-oxycarbonyl, the protecting groups are removed by sequential treatment with ammonia followed by acid, e.g., HCl. Indazole 26 is treated with an appropriate imidate to give 27 and converted to triazole 20 by heating in a polar solvent, e.g., DMF.

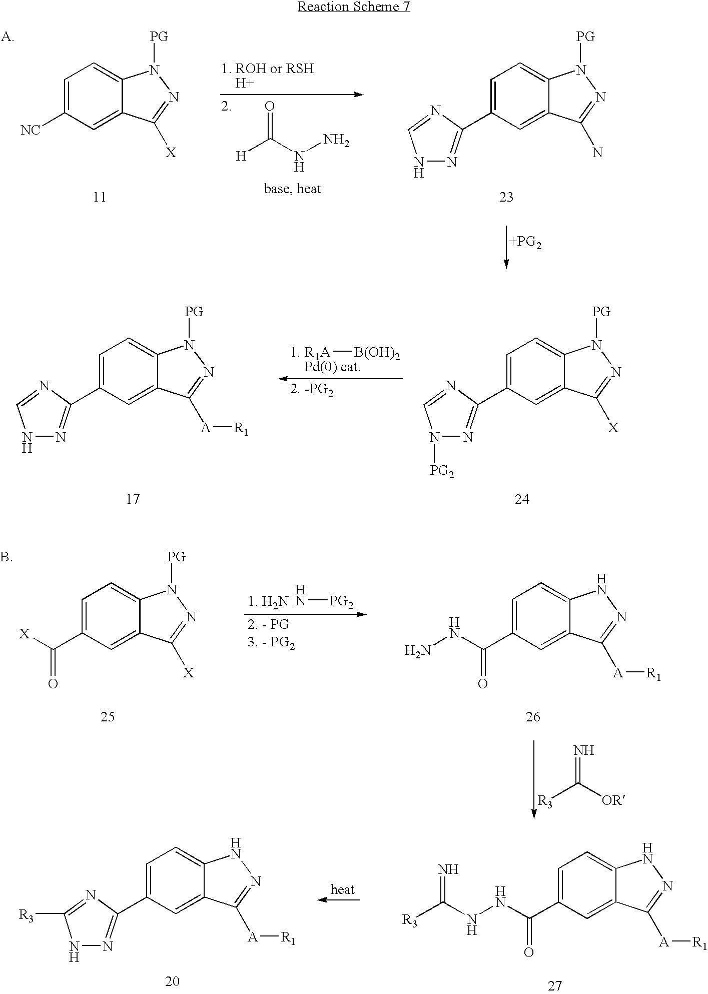

An Indazole Compound can be in the form of a pharmaceutically acceptable salt or a free base. Pharmaceutically acceptable salts of the Indazole Compounds can be formed from organic or inorganic acids. Suitable non-toxic acids include, but are not limited to, inorganic and organic acids such as acetic, alginic, anthranilic, benzenesulfonic, benzoic, camphorsulfonic, citric, ethenesulfonic, formic, fumaric, furoic, galacturonic, gluconic, glucuronic, glutamic, glycolic, hydrobromic, hydrochloric, isethionic, lactic, maleic, malic, mandelic, methanesulfonic, mucic, nitric, pamoic, pantothenic, phenylacetic, phosphoric, propionic, salicylic, stearic, succinic, sulfanilic, sulfuric, tartaric acid, and p-toluenesulfonic acid. Specific non-toxic acids include hydrochloric, hydrobromic, phosphoric, sulfuric, and methanesulfonic acids. The Indazole Compounds can also be used in the form of base addition salts. Suitable pharmaceutically acceptable base addition salts for the Indazole Compounds include, but are not limited to metallic salts made from aluminum, calcium, lithium, magnesium, potassium, sodium and zinc or organic salts made from lysine, N,N′-dibenzylethylenediamine, chloroprocaine, choline, diethanolamine, ethylenediamine, meglumine (N-methylglucamine) and procaine. Examples of specific salts thus include hydrochloride and mesylate salts. Others are well-known in the art, see for example, Remington's Pharmaceutical Sciences, 18th eds., Mack Publishing, Easton Pa. (1990) or Remington: The Science and Practice of Pharmacy, 19th eds., Mack Publishing, Easton Pa. (1995). Thus, the term “pharmaceutically acceptable salt” of an Indazole Compound is intended to encompass any and all acceptable salt forms.

Pharmaceutically acceptable salts of this invention can be formed by conventional and known techniques, such as by reacting a compound of this invention with a suitable acid as disclosed above. Such salts are typically formed in high yields at moderate temperatures, and often are prepared by merely isolating the compound from a suitable acidic wash in the final step of the synthesis. The salt-forming acid can be dissolved in an appropriate organic solvent, or aqueous organic solvent, such as an alkanol, ketone or ester. On the other hand, if the Indazole Compound is desired in the free base form, it can be isolated from a basic final wash step, according to known techniques. For example, a typical technique for preparing hydrochloride salt is to dissolve the free base in a suitable solvent, and dry the solution thoroughly, as over molecular sieves, before bubbling hydrogen chloride gas through it.

The Indazole Compound can also exist in various isomeric forms, including configurational, geometric and conformational isomers, as well as existing in various tautomeric forms, particularly those that differ in the point of attachment of a hydrogen atom. As used herein, the term “isomer” is intended to encompass all isomeric forms of an Indazole Compound, including tautomeric forms of the compound.

The Indazole Compounds can be administered to animals (including humans) orally or parenterally in conventional and well known preparations, such as capsules, microcapsules, tablets, granules, powder, troches, pills, suppositories, injections, suspensions and syrups. Suitable formulations in this regard can be prepared by methods commonly employed using conventional, organic or inorganic additives, such as an excipient (e.g., sucrose, starch, mannitol, sorbitol, lactose, glucose, cellulose, talc, calcium phosphate or calcium carbonate), a binder (e.g., cellulose, methylcellulose, hydroxymethylcellulose, polypropylpyrrolidone, polyvinylprrolidone, gelatin, gum arabic, polyethyleneglycol, sucrose or starch), a disintegrator (e.g., starch, carboxymethylcellulose, hydroxypropylstarch, low substituted hydroxypropylcellulose, sodium bicarbonate, calcium phosphate or calcium citrate), a lubricant (e.g., magnesium stearate, light anhydrous sicilic acid, talc or sodium lauryl sulfate), a flavoring agent (e.g., citric acid, menthol, glycine or orange powder), a preservative (e.g., sodium benzoate, sodium bisulfite, methylparaben or propylparaben), a stabilizer (e.g., citric acid, sodium citrate or acetic acid), a suspending agent (e.g., methylcellulose, polyvinyl pyrrolidone or aluminum stearate), a dispersing agent (e.g., hydroxypropylmethylcellulose), a diluent (e.g., water), and/or a base wax (e.g., cocoa buffer, white petrolatum or polyethylene glycol). The Indazole Compounds can also be administered by any other convenient route, for example, by infusion or bolus injection, by absorption through epithelial or mucocutaneous linings (e.g., oral mucosa, rectal and intestinal mucosa, etc.) and can be administered together with another biologically active agent. Administration can be systemic or local. Various delivery systems are known, e.g., encapsulation in liposomes, microparticles, microcapsules, capsules, etc., and can be used to administer a compound of the invention. In certain embodiments, more than one Indazole Compound is administered to a patient. Methods of administration include but are not limited to intradermal, intramuscular, intraperitoneal, intravenous, subcutaneous, epidural, oral, sublingual, intranasal, intracerebral, intravaginal, transdermal, rectally, by inhalation, or topically, particularly to the ears, nose, eyes, or skin. The preferred mode of administration is left to the discretion of the practitioner, and will depend in-part upon the site of the medical condition. In most instances, administration will result in the release of the Indazole Compound into the bloodstream.

In specific embodiments, it may be desirable to administer one or more Indazole Compound locally to the area in need of treatment. This can be achieved, for example, and not by way of limitation, by local infusion during surgery, topical application, e.g., in conjunction with a wound dressing after surgery, by injection, by means of a catheter, by means of a suppository, or by means of an implant, said implant being of a porous, non-porous, or gelatinous material, including membranes, such as sialastic membranes, or fibers. In one embodiment, administration can be by direct injection at the site (or former site) of an atherosclerotic plaque tissue.

In a particular embodiment, the Indazole Compound is administered by intravenous infusion.

Pulmonary administration can also be employed, e.g., by use of an inhaler or nebulizer, and formulation with an aerosolizing agent, or via perfusion in a fluorocarbon or synthetic pulmonary surfactant. In certain embodiments, the Indazole Compound can be formulated as a suppository, with traditional binders and vehicles such as triglycerides.

In another embodiment, the Indazole Compound can be delivered in a vesicle, in particular a liposome (see Langer, 1990, Science 249:1527-1533; Treat et al, in Liposomes in the Therapy of Infectious Disease and Cancer, Lopez-Berestein and Fidler (eds.), Liss, New York, pp. 353-365 (1989); Lopez-Berestein, ibid., pp. 317-327; see generally ibid.).

In yet another embodiment, the Indazole Compound can be delivered in a controlled release system. In certain embodiments, the Indazole Compound can be delivered in a sustained release or a pulsed release system. In one embodiment, a pump can be used (see Langer, supra; Sefton, 1987, CRC Crit. Ref. Biomed. Eng. 14:201; Buchwald et al., 1980, Surgery 88:507 Saudek et al., 1989, N. Engl. J. Med. 321:574). In another embodiment, polymeric materials can be used (see Medical Applications of Controlled Release, Langer and Wise (eds.), CRC Pres., Boca Raton, Fla. (1974); Controlled Drug Bioavailability, Drug Product Design and Performance, Smolen and Ball (eds.), Wiley, New York (1984); Ranger and Peppas, 1983, J. Macromol. Sci. Rev. MacromoL Chem. 23:61; see also Levy et al., 1985, Science 228:190; During et al., 1989, Ann. Neurol. 25:351; Howard et al., 1989, J. Neurosurg. 71:105). In yet another embodiment, a controlled-release system can be placed in proximity of the target of the Indazole Compound, e.g., the liver, thus requiring only a fraction of the systemic dose (see, e.g., Goodson, in Medical Applications of Controlled Release, supra, vol. 2, pp. 115-138 (1984)). Other controlled-release systems discussed in the review by Langer, 1990, Science 249:1527-1533) can be used.

The present compositions can contain an effective amount of an Indazole Compound, optionally more than one Indazole Compound, preferably in purified form, together with a suitable amount of a pharmaceutically acceptable vehicle so as to provide the form for proper administration to the patient.

In a specific embodiment, the term “pharmaceutically acceptable” means approved by a regulatory agency of the Federal or a state government or listed in the U.S. Pharmacopeia or other generally recognized pharmacopeia for use in animals, and more particularly in humans. The term “vehicle” refers to a diluent, adjuvant, excipient, or carrier with which an Indazole Compound is administered. Such pharmaceutical vehicles can be liquids, such as water and oils, including those of petroleum, animal, vegetable or synthetic origin, such as peanut oil, soybean oil, mineral oil, sesame oil and the like. The pharmaceutical vehicles can be saline, gum acacia, gelatin, starch paste, talc, keratin, colloidal silica, urea, and the like. In addition, auxiliary, stabilizing, thickening, lubricating and coloring agents can be used. When administered to a patient, the Indazole Compound and pharmaceutically acceptable vehicles are preferably sterile. Water is a preferred vehicle when the Indazole Compound is administered intravenously. Saline solutions and aqueous dextrose and glycerol solutions can also be employed as liquid vehicles, particularly for injectable solutions. Suitable pharmaceutical vehicles also include excipients such as starch, glucose, lactose, sucrose, gelatin, malt, rice, flour, chalk, silica gel, sodium stearate, glycerol monostearate, talc, sodium chloride, dried skim milk, glycerol, propyleneglycol, water, ethanol and the like. The present compositions, if desired, can also contain minor amounts of wetting or emulsifying agents, or pH buffering agents.

The present compositions can take the form of solutions, suspensions, emulsion, tablets, pills, pellets, capsules, capsules containing liquids, powders, sustained-release formulations, suppositories, emulsions, aerosols, sprays, suspensions, or any other form suitable for use. In one embodiment, the pharmaceutically acceptable vehicle is a capsule (see e.g., U.S. Pat. No. 5,698,155). Other examples of suitable pharmaceutical vehicles are described in “Remington's Pharmaceutical Sciences” by E. W. Martin.

In a preferred embodiment, the Indazole Compound is formulated in accordance with routine procedures as a pharmaceutical composition adapted for intravenous administration to human beings. Typically, an Indazole Compound for intravenous administration is a solution in sterile isotonic aqueous buffer. Where necessary, the composition can also include a solubilizing agent. Compositions for intravenous administration can optionally include a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the Indazole Compound is to be administered by infusion, it can be dispensed, for example, with an infusion bottle containing sterile pharmaceutical grade water or saline. Where the Indazole Compound is administered by injection, an ampoule of sterile water for injection or saline can be provided so that the ingredients can be mixed prior to administration.

Further, the effect of the Indazole Compound can be delayed or prolonged by proper formulation. For example, a slowly soluble pellet of the Indazole Compound can be prepared and incorporated in a tablet or capsule. The technique can be improved by making pellets of several different dissolution rates and filling capsules with a mixture of the pellets. Tablets or capsules can be coated with a film which resists dissolution for a predictable period of time. Even the parenteral preparations can be made long-acting, by dissolving or suspending the Indazole Compound in oily or emulsified vehicles which allow it to disperse only slowly in the serum.

In a particular embodiment, the Indazole Compound is provided in an aqueous buffered solution.

Compositions for oral delivery can be in the form of tablets, lozenges, aqueous or oily suspensions, granules, powders, emulsions, capsules, syrups, or elixirs, for example. Orally administered compositions can contain one or more optional agents, for example, sweetening agents such as fructose, aspartame or saccharin; flavoring agents such as peppermint, oil of wintergreen, or cherry; coloring agents; and preserving agents, to provide a pharmaceutically palatable preparation. Moreover, where in tablet or pill form, the compositions can be coated to delay disintegration and absorption in the gastrointestinal tract thereby providing a sustained action over an extended period of time. Selectively permeable membranes surrounding an osmotically active driving compound are also suitable for orally administered compounds of the invention. In these later platforms, fluid from the environment surrounding the capsule is imbibed by the driving compound, which swells to displace the agent or agent composition through an aperture. These delivery platforms can provide an essentially zero order delivery profile as opposed to the spiked profiles of immediate release formulations. A time delay material such as glycerol monostearate or glycerol stearate can also be used. Oral compositions can include standard vehicles such as mannitol, lactose, starch, magnesium stearate, sodium saccharine, cellulose, magnesium carbonate, etc. Such vehicles are preferably of pharmaceutical grade.

The amount of an Indazole Compound in a dosage form can differ depending on factors such as, but not limited to, the route by which it is to be administered to patients. However, typical dosage forms of the invention comprise an Indazole Compound in an amount of from about 0.10 mg to about 3500 mg, from about 1 mg to about 2500 mg, from about 10 mg to about 500 mg, from about 25 mg to about 250 mg, from about 50 mg to about 100 mg. Typical dosage forms comprise an Indazole Compound in an amount of about 0.1, 1, 2, 5, 7.5, 10, 12.5, 15, 17.5, 20, 25, 50, 100, 150, 200, 250, 500, 750, 1000, 1500, 2000, 2500, 3000 or 3500 mg. In a particular embodiment, a dosage form comprises an Indazole Compound in an amount of about 1, 2, 5, 10, 25, 50, 100, 250 or 500 mg. In another particular embodiment, a dosage form comprises an amount of about 1, 2, 5, 7, 10, 25, 50, 100, 150, 200, 250, 300, 350, 400, 450 or 500 mg of an Indazole Compound.

In another specific embodiment, a dosage form comprises an aqueous buffered solution (such as a sterile aqueous buffered solution) with a concentration of an Indazole Compound of about 1 mg/mL to about 10 mg/mL, about 1 mg/mL to about 50 mg/mL, or about 1 mg/mL to about 100 mg/mL, for example, 1 mg/mL, 2 mg/mL, 5 mg/mL, 10 mg/mL, 20 mg/mL, 25 mg/mL, 50 mg/mL, 75 mg/mL or 100 mg/mL. In another specific embodiment, provided herein are single unit dosage forms (i.e., a dosage form intended for a single use or single administration) comprising about 1 mg/mL to about 100 mg/mL, for example, 1 mg/mL, 2 mg/mL, 5 mg/mL, 10 mg/mL, 20 mg/mL, 25 mg/mL, 50 mg/mL, 75 mg/mL or 100 mg/mL of an Indazole Compound. A specific, non-limiting example of a single unit dosage form provided herein is a vial comprising 100 mL of a 5 mg/mL buffered, aqueous solution of an Indazole Compound. The buffered, aqueous solution can be sterile and/or suitable for intravenous administration. Of course, it is often practical to administer the daily dose of compound in portions, at various hours of the day. However, in any given case, the amount of Indazole Compound administered will depend on such factors as the solubility of the active component, the formulation used, subject condition (such as weight), and/or the route of administration.

Appropriate doses can be determined by one skilled in the art using known methods (e.g., through a dose-response study). In certain embodiments, an Indazole Compound is administered to a patient in need thereof in single or divided doses of between 0.1 mg/kg and 500 mg/kg, 1 mg/kg and 250 mg/kg, 1 mg/kg and 150 mg/kg, 1 mg/kg and 100 mg/kg, 1 mg/kg and 50 mg/kg, 1 mg/kg and 25 mg/kg, or 1 mg/kg and 10 mg/kg. In one embodiment, an Indazole Compound is administered to a patient in need thereof in a single dose of 1.4 mg/kg, 2.8 mg/kg, 4.7mg/kg or 7.0 mg/kg.

In another embodiment, an Indazole Compound is administered orally to a patient in need thereof in single or divided doses of 200, 300 or 400 mg.

Appropriate dosing regimens can be determined by one skilled in the art using known methods (e.g., through a dose-response study). In certain embodiments, an Indazole Compound is administered to a patient in need thereof daily by intravenous infusion over about 1 hour, about 2 hours, about 4 hours, about 6 hours, about 8 hours, about 10 hours, about 12 hours, about 14 hours, about 16 hours, about 18 hours, about 20 hours, about 22 hours or about 24 hours.

Treatment periods for a course of therapy can span one week, two weeks, three weeks, four weeks, five weeks, six weeks, seven weeks, eight weeks, nine weeks, ten weeks, eleven weeks, twelve weeks, thirteen weeks, fourteen weeks, four months, five months, six months, seven months, eight months, nine months, ten months, eleven months, one year, two years, three years, four years, five years or longer. The treatment periods can be interrupted by periods of rest which can span a day, one week, two weeks, three weeks, four weeks, five weeks, six weeks, seven weeks, eight weeks, nine weeks, ten weeks, eleven weeks, twelve weeks, thirteen weeks, fourteen weeks, four months, five months, six months, seven months, eight months, nine months, ten months, eleven months, one year, two years, three years, four years, five years or longer. In a particular embodiment, a course of therapy comprises administration (e.g., by infusion) an Indazole Compound daily over five consecutive days, followed by a two day break (i.e., no administration of an Indazole Compound), followed by daily administration for an additional five days, followed by another two day break. Such determinations can be made by one skilled in the art (e.g., a physician).

Non-limiting examples of specific dosing regiments include: (1) a 14 day course of treatment comprising intravenous administration of 1.4 mg/kg of an Indazole Derivate to a patient in need thereof over 12 hours q.d. (daily) on days 1-5 and days 8-12, with no administration of an Indazole Compound on days 6, 7, 13 or 14; (2) a 14 day course of treatment comprising intravenous administration of 2.8 mg/kg of an Indazole Derivate to a patient in need thereof over 12 hours q.d. (daily) on days 1-5 and days 8-12, with no administration of an Indazole Compound on days 6, 7, 13 or 14; (3) a 14 day course of treatment comprising intravenous administration of 4.7 mg/kg of an Indazole Derivate to a patient in need thereof over 12 hours q.d. (daily) on days 1-5 and days 8-12, with no administration of an Indazole Compound on days 6, 7, 13 or 14; and (4) a 14 day course of treatment comprising intravenous administration of 7.0 mg/kg of an Indazole Derivate to a patient in need thereof over 12 hours q.d. (daily) on days 1-5 and days 8-12, with no administration of an Indazole Compound on days 6, 7, 13 or 14.

In the embodiments described herein, the amount of, for example, a 5 mg/mL Indazole Compound solution required to administer the proper dose to a patient in need thereof can be determined using the following formula:
X mL=[dose level (mg/kg)×subject weight (kg)]/[5 mg/mL}
wherein X is the volume of the 5 mg/mL Indazole Compound solution necessary to obtain the proper dose.

The following examples are offered by way of illustration, not limitation. (To this end, it should be noted that one or more hydrogen atoms or methyl groups can be omitted from the drawn structures consistent with accepted shorthand notation of such organic compounds, and that one skilled in the art would readily appreciate their presence.)

### 5. EXAMPLES

### Example 1

### 1-(5-(1H-1,2,4-Triazol-5-yl)(1H-indazol-3-yl))-3-(2-piperidylethoxy)benzene

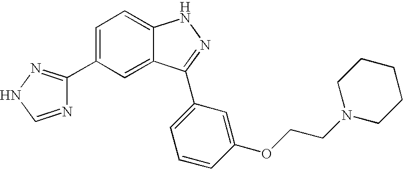

### A. 3-{1-Perhydro-2H-pyran-2-yl-5-[1-(triphenylmethyl)(1,2,4-triazol-3-yl)]-1H-indazol-3-yl}phenol

To a stirred solution of 2-{3-bromo-5-[1-(triphenylmethyl)(1,2,4-triazol-3-yl)]-1H-indazoyl}perhydro-2H-pyran (3.22 g, 5.46 mmol) in dimethoxyethane (27.1 mL) was added 3-hydroxy phenylboronic acid (1.81 g, 8.22 mmol), dichloro[1,1′-bis(diphenylphosphino)ferrocene]palladium (0.447 g, 0.485 mmol), and potassium phosphate (5.78 g, 27.2 mmol) and the mixture was heated at reflux for about 48 h. The mixture was diluted with dichloromethane. The organic extracts were washed with saturated sodium bicarbonate, dried over anhydrous sodium sulfate, filtered and evaporated. Purification of the residue using column chromatography with 20-50% ethyl acetate/hexanes provided the product (3.16 g, 96%, yield). ES-MS (m/z) 362 [M+1(-Tr)]⁺.

### B. 1-(5-(1H-1,2,4-Triazol-5-yl)(1H-indazol-3-yl))-3-(2-piperidylethoxy)benzene

Triphenylphosphine (0.694 g, 2.65 mmol), tetrahydrofuran (2.12 mL), 2-piperidylethanol (0.352 mL, 2.65 mmol) and diethylazodicarboxylate (0.418 mL, 2.65 mmol) were added to 3-{1-perhydro-2H-pyran-2-yl-5-[1-(triphenylmethyl)(1,2,4-triazol-3-yl)]-1H-indazol-3-yl }phenol (0.400 g, 0.662 mmol). The mixture was stirred at ambient temperature for about 23 h and poured into aqueous 6 N hydrochloric acid (30 mL). After stirring at ambient temperature for about 4 h, the mixture was extracted with ether (3×). The aqueous fraction was added to aqueous 6 N sodium hydroxide (30 mL) and the pH adjusted to 11. The solution was extracted with ethyl acetate (3×) and the organic fractions were combined and dried over anhydrous sodium sulfate, filtered and evaporated. The residue was purified using flash chromatography on silica pretreated with 2% triethylamine/ethyl acetate elution followed by 0-20% methanol/ethyl acetate. The desired fractions were concentrated, dissolved in ethyl acetate, washed with aqueous sodium bicarbonate, dried over anhydrous sodium sulfate, filtered and evaporated to provide the title compound (Compound (I)) (0.124 g, 48% yield). ¹H NMR (CD₃OD) δ 8.72 (m, 1H), 8.34 (s, 1H), 8.10 (dd, 1H), 7.67 (dd, 1H), 7.62 (dt, 1H), 7.58 (m, 1H), 7.47 (t, 1H), 7.04 (m, 1H), 4.27 (t, 2H), 2.89 (t, 2.63 (m, 4H), 1.68 (m, 4H), 1.51 (m, 2H). ES-MS (m/z) 389 [M+1]⁺.

### Example 2

### Assays for Measuring Activity of Compounds

The compounds of this invention can be assayed for their activity according to the following procedures.

JNK2 Assay

To 10 μL of the test compound in 20% DMSO/80% dilution buffer consisting of 20 mM HEPES (pH 7.6), 0.1 mM EDTA, 2.5 mM magnesium chloride, 0.004% Triton ×100, 2 μg/mL leupeptin, 20 mM β-glycerolphosphate, 0.1 mM sodium vanadate, and 2 mM DTT in water is added 30 μL of 50 ng His6-JNK2 in the same dilution buffer. The mixture is preincubated for 30 minutes at room temperature. Sixty microliter of 10 μg GST-c-Jun(1-79) in assay buffer consisting of 20 mM HEPES (pH 7.6), 50 mM sodium chloride, 0.1 mM EDTA, 24 mM magnesium chloride, 1 mM DTT, 25 mM PNPP, 0.05% Triton ×100, 11 μM ATP, and 0.5 μCi γ-³²P ATP in water is added and the reaction is allowed to proceed for 1 hour at room temperature. The c-Jun phosphorylation is terminated by addition of 150 μL of 12.5% trichloroacetic acid. After 30 minutes, the precipitate is harvested onto a filter plate, diluted with 50 μL of the scintillation fluid and quantified by a counter. The IC₅₀ values are calculated as the concentration of the test compound at which the c-Jun phosphorylation is reduced to 50% of the control value. Preferred compounds of the present invention have an IC₅₀ value ranging 0.01-10 μM in this assay.

JNK3 Assay

To 10 μL of the test compound in 20% DMSO/80% dilution buffer consisting of 20 mM HEPES (pH 7.6), 0.1 mM EDTA, 2.5 mM magnesium chloride, 0.004% Triton ×100, 2 μmL leupeptin, 20 mM β-glycerolphosphate, 0.1 mM sodium vanadate, and 2 mM DTT in water is added 30 μL of 200 ng His6-JNK3 in the same dilution buffer. The mixture is preincubated for 30 minutes at room temperature. Sixty microliter of 10 μg GST-c-Jun(1-79) in assay buffer consisting of 20 mM HEPES (pH 7.6), 50 mM sodium chloride, 0.1 mM EDTA, 24 mM magnesium chloride, 1 mM DTT, 25 mM PNPP, 0.05% Triton ×100, 11 μM ATP, and 0.5 μCi γ-³²P ATP in water is added and the reaction is allowed to proceed for 1 hour at room temperature. The c-Jun phosphorylation is terminated by addition of 150 μL of 12.5% trichloroacetic acid. After 30 minutes, the precipitate is harvested onto a filter plate, diluted with 50 μL of the scintillation fluid and quantified by a counter. The IC₅₀ values are calculated as the concentration of the test compound at which the c-Jun phosphorylation is reduced to 50% of the control value. Preferred compounds of the present invention have an IC₅₀ value ranging 0.001-10 μM in this assay.

Rat in vivo LPS-induced TNF-α Production Assay

Male CD rats procured from Charles River Laboratories at 7 weeks of age were allowed to acclimate for one week prior to use. A lateral tail vein was cannulated percutaneously with a 22-gage over-the-needle catheter under brief isoflurane anesthesia. Rats were administered test compound either by intravenous injection via the tail vein catheter or oral gavage 15 to 180 min prior to injection of 0.05 mg/kg LPS (E. Coli 055:BS). Catheters were flushed with 2.5 mL/kg of normal injectable saline. Blood was collected via cardiac puncture 90 minutes after LPS challenge. Plasma was prepared using lithium heparin separation tubes and frozen at −80° C. until analyzed. TNF-α levels were determined using a rat specific TNF-α ELISA kit (Biosource). The ED₅₀ values are calculated as the dose of the test compound at which the TNF-α production is reduced to 50% of the control value. Preferred compounds of the present invention have an ED₅₀ value ranging 1-30 mg/kg in this assay.

### Example 3

### Dosing Regimen

Suitable doses and dosing regimens can be determined by those skilled in the art, such as a physician. In one embodiment, the following dosing regimen is used to administer an Indazole Compound to a patient in need thereof.

An Indazole Compund is provided as 100 mL of a 5 mg/mL buffered, aqueous solution. A patient in need thereof is administered either 1.4 mg/kg, 2.8 mg/kg, 4.7 mg/kg or 7.0 mg/kg of the Indazole Compound daily over 12 hours as an intravenous infusion for five consecutive days. This is followed by a two day break. The patient in need thereof is then again administered either 1.4 mg/kg, 2.8 mg/kg, 4.7 mg/kg or 7.0 mg/kg of the Indazole Compound daily over 12 hours as an intravenous infusion for five more consecutive days. This is then followed by another two day break. The amount of the 5 mg/mL Indazole Compound solution required to administer the proper dose to a patient in need thereof can be determined using the following formula:
X mL=[dose level (mg/kg)×subject weight (kg)]/[5 mg/mL}
wherein X is the volume of the 5 mg/mL Indazole Compound solution necessary to obtain the proper dose. This course of treatment can be repeated as many times as is determined to be necessary by one skilled in the art (e.g., a physician). Bone marrow aspiration and/or biopsy can be performed following administration of the Indazole Compound to determine efficacy.

It will be appreciated that, although specific embodiments of the invention have been described herein for purposes of illustration, various modifications can be made without departing from the spirit and scope of the invention. Such modifications are intended to fall within the scope of the appended claims.

A number of references have been cited, the entire disclosures of which are incorporated herein by reference.

What is claimed is:

1. A method for treating acute myelogenous leukemia comprising administering to a patient in need thereof an effective amount of a compound having the structure: 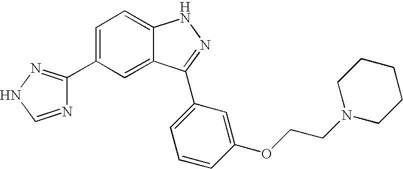
or a pharmaceutically acceptable salt thereof.

2. The method of claim 1, wherein the compound is administered as a pharmaceutical composition.

3. The method of claim 1, wherein the compound is administered orally.

4. The method of claim 1, wherein the compound is administered parenterally.

5. The method of claim 4, wherein the compound is administered intravenously, intramuscularly, intradermally or subcutaneously.

6. The method of claim 5, wherein the compound is administered intravenously.

7. The method of claim 6, wherein the compound is administered by infusion.

8. The method of claim 6, wherein the compound is administered daily.

9. A method for preventing acute myelogenous leukemia comprising administering to a patient in need thereof an effective amount of a compound having the structure: 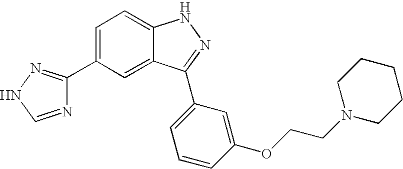
or a pharmaceutically acceptable salt thereof.

10. The method of claim 9, wherein the compound is administered as a pharmaceutical composition.

11. The method of claim 9, wherein the compound is administered orally.

12. The method of claim 9, wherein the compound is administered parenterally.

13. The method of claim 12, wherein the compound is administered intravenously, intramuscularly, intradermally or subcutaneously.

14. The method of claim 13, wherein the compound is administered intravenously.

15. A pharmaceutical composition comprising 1 mg/mL to 10 mg/mL of a compound having the structure: 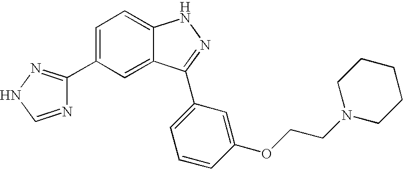
or a pharmaceutically acceptable salt thereof.

16. The pharmaceutical composition of claim 15, wherein the composition is in liquid form.

17. The pharmaceutical composition of claim 16, wherein the composition is aqueous.

18. The pharmaceutical composition of claim 16, wherein the composition is sterile.

19. A single unit dosage form suitable for intravenous administration comprising 5 mg/mL of a compound having the structure: 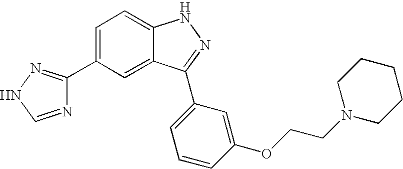
or a pharmaceutically acceptable salt thereof.

20. The single unit dosage form of claim 19, wherein the dosage form is aqueous.
